(19) United States
(12) Patent Application Publication
(10) Pub. No.: US 2026/0262983 A1
(43) Pub. Date: Sep. 10, 2026

(54) ACE EFFICIENCY: WEARABLE SYSTEM FOR TASK-CRITICAL PERFORMANCE READINESS

(71) Applicant: Kristy Violette, Sarasota, FL (US)

(72) Inventor: Kristy Violette, Sarasota, FL (US)

(21) Appl. No.: 19/194,650

(22) Filed: Apr. 30, 2025

Related U.S. Application Data
(60) Provisional application No. 63/768,527, filed on Mar. 7, 2025.

(51) Int. Cl. A61B 5/16; A61B 5/00; A61B 5/0205; A61B 5/024; A61B 5/0533; A61B 5/11; A61B 5/1455; A61B 5/397; G06Q 10/0639

(52) U.S. Cl. CPC A61B 5/18; A61B 5/0002; A61B 5/0205; A61B 5/165; A61B 5/486; A61B 5/681; A61B 5/7207; A61B 5/7246; A61B 5/725; A61B 5/7264; A61B 5/746; B60K 28/02; G06Q 10/06398; A61B 5/02405; A61B 5/0533; A61B 5/11; A61B 5/14552; A61B 5/397; A61B 2503/22; A61B 2503/40; A61B 2562/0219

(57) ABSTRACT

Ace Efficiency evaluates performance readiness in high-stakes roles such as healthcare, aviation, and tactical operations using a wearable device with biometric sensors (PPG, SpO₂, GSR, EMG, motion). The method uses short, user-initiated check-ins to capture a physiological snapshot, generating an efficiency score via a role-specific weighting algorithm compared to a personalized baseline. It categorizes scores into feedback tiers (proceed, recover, escalate), delivers interventions, stores encrypted logs for compliance, and synchronizes performance between a user and their working animal using biometric synchrony (Pearson correlation coefficient) for shared feedback. A system includes a processor to execute the algorithm, calculate an adaptive threshold, and deliver tiered outputs via haptic, auditory, or visual means.

### CROSS-REFERENCE TO RELATED APPLICATIONS

This application claims priority to U.S. Provisional Patent Application No. 63/768,527, filed Mar. 7, 2025, titled “Wearable Performance Monitoring System with Ace AI Assistant,” and U.S. Provisional Patent Application No. 63/788,032, filed Apr. 13, 2025, titled “A system and method for evaluating performance readiness in task-critical, high-stakes roles using episodic biometric check-ins, adaptive scoring algorithms, and role-specific feedback. A wearable device captures multi-sensor biometric data—including photoplethysmography (PPG), SpO₂, galvanic skin response (GSR), electromyography (EMG), and motion via accelerometer and gyroscope—compared against a personalized baseline established through at least five non-continuous, user-initiated or scheduled check-ins over a seven-day calibration period. An efficiency score is categorized into feedback tiers (proceed, recover, escalate) based on deviations from adaptive thresholds tailored to role-specific fatigue trends and environmental stressors. Real-time interventions or supervisor alerts are triggered as needed. An Alpha Mode synchronizes a human and service animal using a Pearson correlation algorithm for co-regulation. A Rebalance Mode supports recovery from burnout or relapse with non-medicated interventions. The system ensures privacy through non-continuous monitoring, predicted to reduce fatigue-related errors by 15% in high-stakes roles, per conceptual simulations and algorithmic modeling (see Page 8).”

### BACKGROUND OF THE INVENTION

Wearable technology has advanced health and wellness, with devices from Apple, Fitbit, and WHOOP tracking sleep and activity. However, these tools prioritize recovery and general health, not moment-to-moment readiness for task-critical roles like aviation, surgery, military, or emergency response. Prior efforts, such as Allstate's driver readiness system (U.S. Pat. No. 10,446,026B2), used behavioral check-ins to prevent unsafe vehicle operation, lacking multi-sensor biometrics or adaptive feedback. Similarly, exertion-focused systems like U.S. Pat. No. 11,202,654B2 (Metabolic Check-In) employed episodic biometric testing but without role-specific calibration or escalation protocols. Unlike episodic biometric testing systems such as U.S. Pat. No. 11,202,654B2 (Metabolic Check-In), which lack defined sampling windows and role-specific calibration, the present invention uses a 30-second window for time-aligned biometric samples during user-initiated or scheduled check-ins, ensuring privacy and tailored performance assessment for high-stakes roles (see Page 8). Vibration-based wearables, such as Feelmore Labs' Cove (U.S. Pat. No. 10,786,666), targeted individual stress relief via neural stimulation, omitting multi-user coordination. Systems like Hexoskin (CA2896498C) monitor biometrics for fitness but lack performance optimization for high-stakes roles. No existing wearable facilitates synchronized physiological regulation between a human and a service animal or offers adaptive, role-weighted feedback based on real-time biometrics and predictive thresholds. Unlike continuous monitoring systems, Ace Efficiency's episodic check-ins prioritize user privacy and regulatory compliance, such as FAA audits. The present invention addresses these gaps with a system for real-time performance readiness evaluation, building on U.S. Provisional Application No. 63/768,527, which introduced a wearable check-in model for mental health, and No. 63/788,032, which added co-regulation and recovery extensions.

### BRIEF SUMMARY OF THE INVENTION

Ace Efficiency is a structured biometric performance evaluation system designed for high-responsibility environments such as healthcare, aviation, and tactical operations. Unlike traditional wellness wearables that rely on continuous monitoring or population-level thresholds, Ace Efficiency utilizes short, user-initiated check-ins to capture a real-time snapshot of physiological readiness and generate a performance efficiency score.

The invention provides a unified method for evaluating performance readiness in high-stakes roles, encompassing users and their working animals as integral high-responsibility participants in such environments. The method uses wearable devices with biometric sensors to assess readiness, whether for a single participant or in synchronization between a user and their working animal, ensuring optimal performance through a shared role-specific weighting algorithm and feedback mechanisms.

### BRIEF DESCRIPTION OF DRAWINGS

FIG. 1 illustrates a wearable device with a wristband housing GSR, EMG, and motion sensors (accelerometer and gyroscope), and a top casing containing PPG and SpO2 sensors, arranged for biometric data collection; wristband placement boosts EMG stability for surgical use.

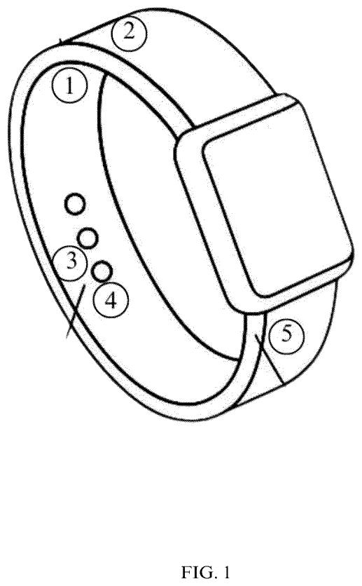

FIG. 2 depicts a flowchart of the feedback escalation process, showing tiered outputs (proceed, delay, recover, escalate) with a 15-min recover loop (R1) and a supervisor alert pathway (E1) from escalate.

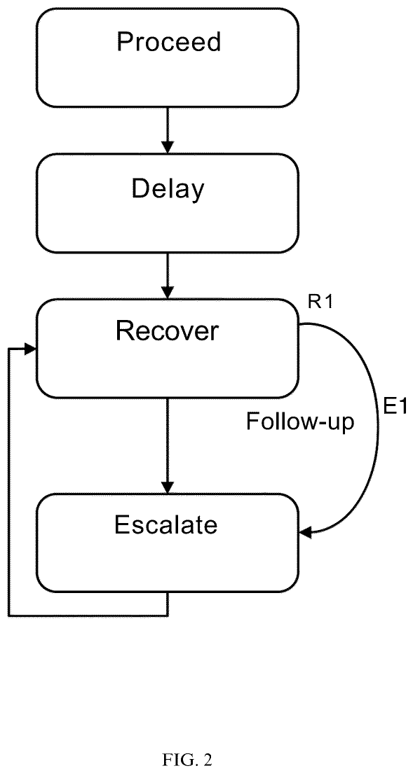

FIG. 3 shows a block diagram of the system architecture, integrating the core system with Alpha Mode and Rebalance Mode modules.

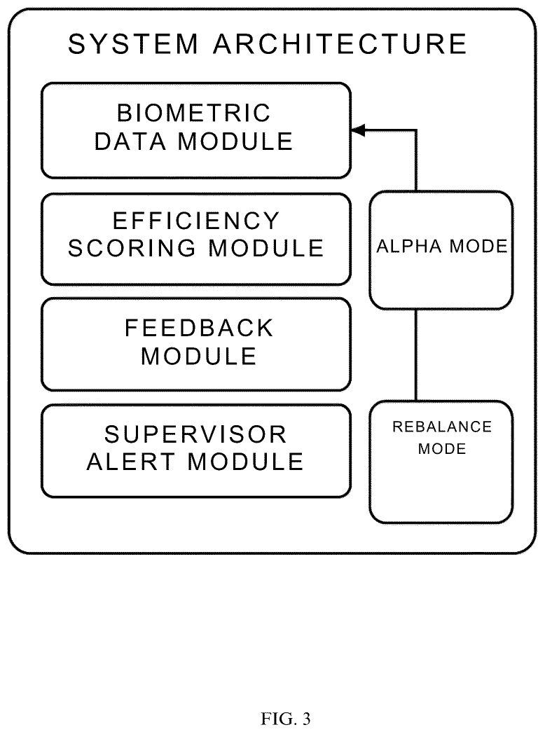

FIG. 4 presents a bar graph comparing role-specific biometric weights for pilot and surgeon profiles, illustrating dynamic weight adjustments for SpO2, HRV, EMG, GSR, and motion sensors in percentages.

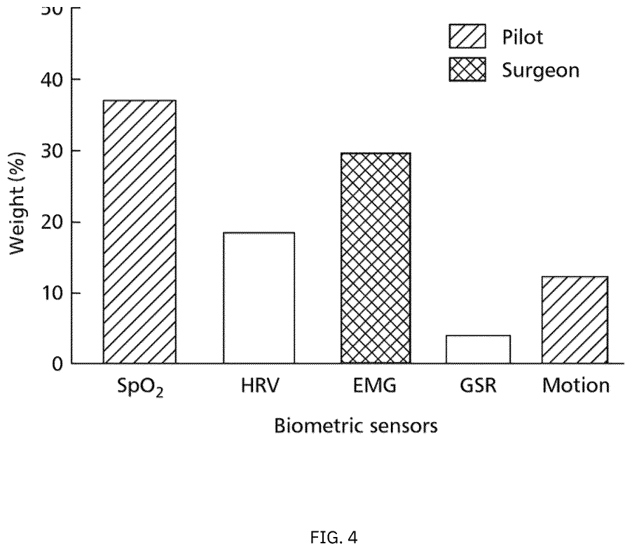

FIG. 5 illustrates a supervisor dashboard displaying an efficiency score of 66% and heart rate of 92 bpm, as described on Page 13.

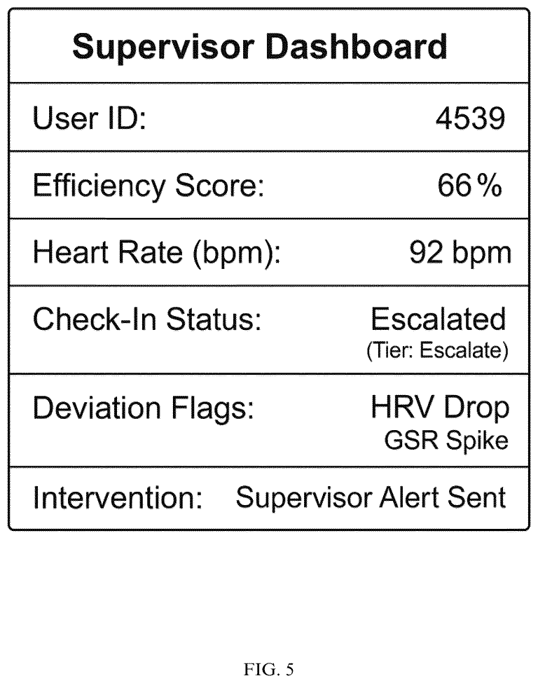

### DETAILED DESCRIPTION

Ace Efficiency is a biometric performance monitoring system built for high-risk roles where preventable error must be intercepted before it occurs. Unlike traditional wearables that rely on continuous background tracking or generalized health norms, Ace Efficiency introduces a structured, episodic check-in method that captures five cross-domain physiological signals—autonomic (PPG), neuromuscular (EMG), emotional (GSR), respiratory (SpO₂), and postural (accelerometer/gyroscope)—within a short, user-initiated session. These readings are processed through a novel scoring engine that compares users and their working animals not to healthcare-derived thresholds, but to their own individual personal best—creating a dynamic model that reinforces high performance rather than punishing variance from population averages. Sensor architecture is intentionally zoned, with optical sensors embedded in the device housing and contact-based sensors integrated into the wearable band, enabling high-fidelity signal fusion during short-use sessions. Scores trigger tiered readiness classifications (Cleared/Caution/Impaired), which may include gatekeeping logic, escalated alerts, and logged decisions. Adaptive weighting based on task profile—e.g., fine motor vs cognitive precision—ensures context-relevant accuracy. Additional modular expansions, including Rebalance Mode for continuous recovery monitoring and Ace Alpha for multispecies team readiness, further distinguish Ace Efficiency as a real-time decision support tool designed not to track fitness—but to prevent critical failure.

Ace Efficiency is a structured biometric performance evaluation system designed for high-responsibility environments such as healthcare, aviation, and tactical operations. Unlike traditional wellness wearables that rely on continuous monitoring or population-level thresholds, Ace Efficiency utilizes short, user-initiated check-ins to capture a real-time snapshot of physiological readiness and generate a performance efficiency score.

Ace Efficiency's novelty is defined by the following:
A check-in based method that replaces continuous passive monitoring with structured, episodic biometric analysis
Multi-domain sensor fusion across autonomic, neuromuscular, emotional, respiratory, and postural systems
Zoned sensor placement optimizing signal fidelity across screen and band regions
Personalized baselines anchored to each participant's historical peak state rather than clinical averages
Tiered feedback response system that transforms raw scores into actionable recommendations and escalation logic
Adaptability across user roles and environments, supporting both professional customization and safety-based gating

These structural and functional elements collectively define Ace Efficiency as a novel system for task-specific risk prevention and readiness validation.

### 2. System Components

The Ace Efficiency system includes a wearable housing equipped with the following:
A display-integrated housing zone with optical sensors (PPG, SpO₂)
A band-integrated zone with contact sensors (EMG, GSR) and motion sensors (accelerometer/gyroscope)
A processor configured to receive, analyze, and compare biometric data
A user interface for displaying score outcomes and recommended actions
Optional communication modules for supervisory notifications

### 3. Sensor Function and Placement

Ace Efficiency employs five key biometric sensors, each mapped to a distinct physiological domain:
PPG—autonomic nervous system regulation
EMG—neuromuscular fatigue
GSR—emotional and sympathetic stress
SpO₂—respiratory and cognitive oxygenation
Accelerometer/Gyroscope—postural control and fatigue drift

Sensors are intentionally separated into functional zones to optimize signal fidelity during short-duration check-ins. This zoned layout provides structural novelty not seen in general-purpose smartwatches or wellness devices.

### 4. Summary

Ace Efficiency is a structured biometric performance evaluation system designed for high-responsibility environments such as healthcare, aviation, and tactical operations. Unlike traditional wellness wearables that rely on continuous monitoring or population-level thresholds, Ace Efficiency utilizes short, user-initiated check-ins to capture a real-time snapshot of physiological readiness and generate a performance efficiency score.

The invention provides a unified method for evaluating performance readiness in high-stakes roles, encompassing users and their working animals as integral high-responsibility participants in such environments. The method uses wearable devices with biometric sensors to assess readiness, whether for a single participant or in synchronization between a user and their working animal, ensuring optimal performance through a shared role-specific weighting algorithm and feedback mechanisms.

### 5. Scoring Logic

During each check-in, all sensors activate simultaneously. The processor evaluates current readings against the participant's individualized baseline using a weighted algorithm. The resulting performance efficiency score reflects the participant's total functional readiness in that moment, factoring in emotional, physical, respiratory, and neuromuscular domains. The composite efficiency score is calculated as a weighted average of the biometric signals, expressed as a percentage deviation from the participant's baseline, with specific weights assigned to each signal as described in Section 12.

### 6. Baseline Establishment and Personalization

Ace Efficiency establishes a personalized baseline during an initial calibration phase, typically consisting of at least three to seven check-in sessions. This baseline is updated over time using biometric trend analysis. Crucially, Ace Efficiency does not compare participants to generalized medical standards, but instead tracks deviation from their own historical peak state—ensuring a personalized, fair, and adaptive readiness score. This “personal best” model is rare and adds a strong differentiating angle.

### 7. Feedback Output and Escalation Protocol

If the check-in score is ≥70% of baseline, the participant is cleared. If the score is <70%, an Ace Action is triggered based on sensor readings (e.g., breath work, hydration). The participant then re-checks after recovery. If performance remains low, an escalated action is delivered. If unresolved, a supervisor alert is sent. This tiered logic converts raw data into real-time, risk-reducing decisions.

### 8. Customization and Professional Use

Thresholds and escalation timing may be customized by organization or role. A surgical team may use a higher cutoff, while logistics roles may tolerate broader variance. This adaptability makes Ace Efficiency applicable to military, healthcare, law enforcement, emergency response, and aviation sectors.

### 9. Functional Benefits and Use Case Differentiation

Ace Efficiency improves performance readiness of users and their working animals in high-responsibility roles while reducing preventable error rates in mission-critical tasks. By identifying fatigue, stress, or impairment before critical decisions are made, the system protects public safety and participant well-being. This use-case driven logic places Ace Efficiency far beyond general fitness trackers or lifestyle wearables.

### 10. Structural and Method-Based Novelty

Ace Efficiency is novel in both structure and method:
The use of multi-domain sensor fusion in a short-duration check-in window is not seen in existing systems.
Zoned sensor placement enables accurate episodic readings and enhances signal quality.
Feedback output is actionable and escalates logically, unlike generalized wellness scores.
Personalized scoring based on participant-specific historical peak performance replaces traditional healthcare thresholds.

This combination of features makes Ace Efficiency a unique, patentable solution for performance management in high-risk fields.

### 11. Advantages of the Invention

Ace Efficiency offers a novel approach to biometric monitoring, prioritizing structured check-ins, multi-domain signal fusion, personalized baseline adaptation, and tiered decision logic. It is optimized for real-time readiness assessment and error prevention, with applications in both civilian and defense sectors. No existing system delivers this level of specificity, adaptability, or real-world applicability.

### 12. Simulated Dataset Demonstrating Ace Efficiency Logic

To illustrate the internal logic and structured escalation method of the Ace Efficiency system, a representative simulated dataset was generated using values aligned with publicly available biometric sensor ranges and research-backed behavioral patterns. The mock data simulates a single user during both their baseline establishment phase and subsequent performance check-ins.

Each biometric check-in consists of five physiological inputs captured during a user-initiated session:
PPG—pulse waveform patterns and heart rate variability (HRV), indicating autonomic readiness
EMG—neuromuscular tension and fatigue levels
GSR—emotional stress reactivity via skin conductance
SpO₂—oxygen saturation affecting cognitive clarity
Accelerometer/Gyroscope—postural control and subtle movement instability

The initial seven sessions are designated as baseline calibration events. Their values are intentionally clustered around 100, which in the Ace Efficiency framework represents the individual participant's own historical peak performance. Unlike systems that compare against clinical norms, Ace Efficiency scores are calculated as a percentage of this personal best to deliver fair, individualized insights.

### 13. Scoring Formula and Metric Weighting

The Ace Efficiency system calculates a composite efficiency score for each check-in session by averaging the weighted contribution of each biometric signal. Each domain impacts a unique aspect of readiness, and the system's default algorithm weights each domain as follows:
PPG (HRV and waveform): 25%—representing stress regulation and autonomic recovery
EMG (neuromuscular tension): 20%—linked to physical strain and motor precision
GSR (skin conductance): 20%—emotional regulation and pressure response
SpO₂ (oxygen saturation): 20%—cognitive clarity and alertness
Accelerometer/Gyroscope: 15%—physical balance and postural fatigue

The composite efficiency score is calculated as a weighted average, expressed as a percentage deviation from the participant's baseline:

$$Composite Efficiency Score = ( PPG\_Score * 0.25 ) + ( EMG\_Score * 0.2 ) + ( GSR\_Score * 0.2 ) + ( {SpO}_{2} Score * 0.2 ) + ( Motion\_Score * 0.15 )$$

where each Score (e.g., PPG_Score, EMG_Score) is a normalized value (0-100) representing the deviation of the current reading from the participant's baseline for that biometric signal.

These weights were selected based on performance science literature linking each signal to task-critical readiness. Scores are aggregated, converted into a single efficiency percentage, and compared against the participant's personal baseline. This model supports intervention before critical performance degradation risks, such as burnout, trauma, or relapse, manifest in high-stakes roles, using specific biometric thresholds (e.g., HRV reduction greater than 10% or GSR elevation greater than 15 microsiemens over two non-continuous sessions within a 24-hour period, as detailed in Section 14).

The Decay_Factor is a role-specific multiplier, established during baseline calibration, that adjusts the sensitivity of fatigue detection according to operational risk profiles.

### 14. Adaptive Scoring Weight Models by Task Type

Ace Efficiency supports adaptive scoring based on the participant's operational context. Upon account setup or institutional configuration, the participant's role is identified and internally mapped to one of several performance-demand categories. Each category is associated with a unique biometric weighting model that determines how efficiency scores are calculated. This logic ensures that performance metrics most critical to the participant's actual duties carry proportionally greater influence on their overall readiness score.

For example, a neurosurgeon may be mapped to a ‘fine motor dominant’ category, where EMG data carries greater influence (e.g., EMG at 40%, GSR at 20%, SpO₂ at 40%) due to the precision and endurance required for surgical procedures. A commercial airline pilot or air traffic controller may fall under a ‘cognitive precision’ category, emphasizing SpO₂ and PPG (e.g., SpO₂ at 40%, HRV at 35%, motion at 25%) to reflect the importance of mental clarity, sustained focus, and autonomic regulation. A ride share driver may be mapped to a ‘postural and emotional load’ category, where GSR and accelerometer inputs are prioritized to detect fatigue, frustration, or subtle physical strain during extended driving shifts.

This abstraction allows the system to function seamlessly across medical, tactical, transportation, and administrative domains without requiring technical calibration or sensor configuration by the end user. Role-to-category mapping is handled within the system logic and may be institutionally managed or user-selected at setup.

### 15. Modular Extensions-Ace Rebalance and Ace Alpha

Ace Rebalance is a modular extension of the Ace Efficiency system, designed to operate in continuous mode during recovery or rehabilitation phases. While the core Ace Efficiency system utilizes episodic check-ins, Rebalance Mode provides real-time monitoring for early signs of behavioral decline, physiological imbalance, or emotional dysregulation. This mode is particularly useful for relapse prevention in individuals recovering from substance use disorders.

Using the same sensor array, Ace Rebalance identifies patterns of physiological stress, fatigue, or emotional volatility, and delivers supportive interventions such as hydration prompts, breathwork guidance, micronutrient recommendations, or grounding cues. It can be adapted to provide substance-specific support—such as magnesium or B-complex prompts for alcohol recovery or movement reminders for stimulant withdrawal—and logs efficiency improvements over time to visually affirm user progress.

Ace Rebalance may optionally trigger notifications to a care team or support network when patterns suggest elevated relapse risk, such as an HRV reduction greater than 10% or a GSR elevation greater than 15 microsiemens (μS) over two non-continuous check-in sessions within a 24-hour period. These alerts are customizable and privacy-preserving, designed to increase support without stigmatizing the user.

Ace Alpha is a species-inclusive module that extends the core Ace Efficiency scoring and readiness logic to teams composed of users and working animals. This may include working animals in roles such as search and rescue, military operations, law enforcement, or daily support functions such as autism assistance or visual guidance. In the context of Ace Alpha, the user acts as the handler of the working animal, forming a team in high-responsibility roles.

The system enables parallel biometric monitoring of the user and their working animal, creating a co-regulatory loop that enhances team safety and synchronization. Signals from each party are evaluated for biometric synchrony using a Pearson correlation coefficient (r) over a 10-second window, with motion artifacts mitigated through low-pass filtering at 5 Hz. The Pearson correlation coefficient is calculated as:

$$r = Σ [ ( x\_i - \overline{x} ) ( y\_i - \overline{y} ) ] / √ [ {Σ ( x\_i - \overline{x} )}^{2} * {Σ ( y\_i - \overline{y} )}^{2} ]$$

where x_i and y_i are paired biometric signals (e.g., HRV, motion) from the user and working animal, respectively, over the 10-second window, and x̅ and y̅ are the means of those signals. A deviation from biometric synchrony is determined when the Pearson correlation coefficient falls below an adaptive threshold of r=0.7±0.1. The adaptive threshold is dynamically configured based on species-specific baseline behavioral norms, user calibration inputs during setup, and real-time biometric feedback and historical data trends. Upon detecting a deviation, shared feedback outputs, such as rest prompts, environmental changes, or mutual task delay, are delivered to the respective wearable devices to promote co-regulation and restoration of biometric synchrony.

Ace Alpha promotes interspecies situational awareness, reinforcing the concept that performance, trust, and wellness must flow in both directions across the team. This extension supports a novel class of multispecies performance monitoring tools built on the same foundation as the core Ace Efficiency engine.

### 16. Conclusion and Filing Notes

The Ace Efficiency system is a novel, user-personalized performance monitoring platform that leverages episodic biometric check-ins, dynamic scoring logic, and task-specific response frameworks to reduce preventable errors and optimize readiness in high-responsibility roles.

Its modular architecture enables adaptive logic for role-based scoring, continuous recovery monitoring through Rebalance Mode, and multispecies team synchronization through Ace Alpha. Together, these components define a next-generation biometric platform capable of enhancing safety, performance, and well-being across a wide range of users and their working animals in high-responsibility roles.

The details presented in this specification are intended to support a utility patent application by demonstrating system enablement, structural novelty, scoring adaptability, and real-world application through simulated data and example use cases. Claims have been separately prepared to reflect the core method, system components, adaptive personalization, and tiered feedback architecture.

1. A method implemented by a wearable device and a processing system for evaluating readiness of a user in a high-responsibility role within a safety-critical environment, wherein the high-responsibility role comprises at least one of piloting an aircraft, performing surgery, or managing tactical operations, the method comprising: initiating, prior to commencement of performance of the high-responsibility role, a user-triggered episodic biometric check-in session of predetermined limited duration using a wearable device worn by the user, wherein, outside the biometric check-in session, biometric sensors of the wearable device are maintained in a non-sampling state such that physiological data acquisition is limited to the user-triggered biometric check-in sessions and substantially no physiological data is acquired or stored between biometric check-in sessions; capturing, during the biometric check-in session, physiological signals from: (i) at least one stress-related biometric sensor of the wearable device configured to measure heart rate or heart rate variability of the user, (ii) at least one neuromuscular sensor of the wearable device configured to measure electromyography signals of the user, and (iii) at least one oxygenation sensor of the wearable device configured to measure blood oxygen saturation of the user; for each of the physiological signals, computing, at the wearable device or at the processing system communicatively coupled to the wearable device, a deviation value relative to an individualized baseline profile associated with the user, the individualized baseline profile being derived from physiological data collected from a plurality of prior biometric check-in sessions in which the user was determined to be ready for the high-responsibility role; combining the deviation values according to role-specific predetermined weighting factors stored in a memory, wherein the weighting factors are based on empirical correlations to user readiness and sum to 1.0, to generate a composite efficiency score; comparing the composite efficiency score to a plurality of threshold values defining at least (i) an optimal range, (ii) a cautionary range, and (iii) an impaired range, to determine a readiness classification level selected from at least an optimal state, a cautionary state, and an impaired state; and providing a feedback output comprising a clearance status responsive to said readiness classification to implement safety-based gating of the high-responsibility role, wherein the clearance status comprises at least one of: (a) a proceed indication authorizing commencement of the high-responsibility role when the readiness classification level is in the optimal state, (b) a recover indication requiring the user to complete a recovery protocol and undergo a subsequent biometric check-in session prior to commencement when the readiness classification level is in the cautionary state or the impaired state, or (c) an escalate indication triggering notification to supervisory personnel when the readiness classification level is in the impaired state, thereby intercepting potential user error before the high-responsibility role is performed.

2. The method of claim 1, wherein the feedback output includes a categorized performance tier selected from optimal, cautionary, or impaired.

3. The method of claim 2, further comprising triggering a notification to a supervisor dashboard when said readiness classification falls below a defined safety margin for two consecutive check-in sessions.

4. The method of claim 1, wherein generating the composite efficiency score includes applying a role-specific weighting algorithm to the physiological signals, the role-specific weighting algorithm assigning weights to each signal based on a high-stakes category associated with the user, wherein the high-stakes category includes at least one of aviation or surgery, and wherein weights for aviation roles include SpO2 at 40%, heart rate variability (HRV) at 35%, and motion at 25%, and weights for surgical roles include EMG at 40%, GSR at 20%, and SpO2 at 40%.

5. The method of claim 1, further comprising evaluating coordinated biometric patterns across multiple sensor modalities of the wearable device over a predetermined window and determining a synchrony deviation when a correlation between the modalities falls below an adaptive threshold dynamically configured based on empirical data and historical performance of the user; and delivering a feedback output through output channels of the wearable device to promote restoration of coordinated biometric patterns.

6. The method of claim 5, wherein delivering the feedback output comprises providing a pulse, auditory cue, or visual signal through the output channels of the wearable device, with intensity and frequency of the feedback output adjusted based on the degree of synchrony deviation.

7. A wearable device system comprising: a housing containing optical sensors for photoplethysmography (PPG) and blood oxygen saturation (SpO2), the housing configured to isolate optical signals; a wristband with skin-contact sensors for galvanic skin response (GSR), electromyography (EMG), and motion sensors comprising accelerometer and gyroscope, the wristband configured to optimize skin-contact signal fidelity during short-duration check-ins; a processor comprising modules configured to: execute a role-specific weighting algorithm, wherein the modules are configured to dynamically adjust biometric data weights based on task performance error rates from standardized simulated tasks; calculate an adaptive threshold according to the formula: Threshold=Baseline_Average−(Decay_Factor*Fatigue_Index), where Fatigue_Index is determined by: Fatigue_Index=(0.4*HRV_Reduction)+(0.3*GSR_Elevation)+(0.3*Motion_Irregularity); store encrypted logs of biometric check-ins compliant with cybersecurity standards such as NIST 800-53; a wireless communication module for secure alert transmission; and a feedback module for delivering real-time tiered outputs to the user via haptic, auditory, or visual means.

8. The method of claim 1, wherein the non-sampling state is maintained by actively disabling biometric data acquisition between user-triggered biometric check-in sessions, with no physiological data acquired or stored outside the biometric check-in sessions.

9. The method of claim 1, further comprising detecting early indicators of at least one of burnout, trauma, or relapse based on at least one of an HRV reduction greater than 10% or a GSR elevation greater than 15 microsiemens over two non-continuous check-in sessions within a 24-hour period; and triggering a tailored intervention comprising at least one of a hydration cue or a breathwork prompt.

10. The method of claim 3, further comprising comparing the composite efficiency score to an adaptive threshold to inform the readiness classification level, and logging a supervisor override of the notification for a critical task performance if the composite efficiency score remains below the adaptive threshold, wherein the logging ensures compliance and transparency.

11. The method of claim 9, wherein a follow-up readiness biometric check-in session is required within a predetermined time period following an output indicating that the readiness classification level is in the cautionary state or the impaired state, and wherein the predetermined time period is adjusted based on detection of a stabilizing HRV trend comprising a variance of less than 5% from the individualized baseline profile over a 30-second measurement window.

12. The method of claim 1, wherein the plurality of prior biometric check-in sessions from which the individualized baseline profile is derived comprises exclusively sessions in which: (i) the composite efficiency score exceeded a predetermined readiness threshold at the time of the check-in session, and (ii) the user subsequently completed the high-responsibility role without a reported safety incident, such that the individualized baseline profile represents physiological parameters associated with verified successful task performance.

13. The method of claim 1, wherein the escalate indication is triggered only when the readiness classification level is in the impaired state for at least two consecutive biometric check-in sessions within a predetermined time window, thereby reducing false-positive escalations from transient physiological fluctuations.
